(19) United States
(12) Patent Application Publication
(10) Pub. No.: US 2026/0283512 A1
(43) Pub. Date: Sep. 24, 2026

(54) MICROFLUIDIC PAPER-BASED ANALYTICAL DEVICES FOR QUANTIFICATION OF ANALYTES IN INTERSTITIAL FLUID

(71) Applicant: Trustees of Tufts College, Medford, MA (US)

(72) Inventors: Sameer SONKUSALE, Lincoln, MA (US); Danilo Martin DOS SANTOS, Medford, MA (US); Kawin KHACHORNSAKKUL, Malden, MA (US)

(21) Appl. No.: 19/566,378

(22) Filed: Mar. 13, 2026

Related U.S. Application Data
(60) Provisional application No. 63/773,744, filed on Mar. 18, 2025.

(51) Int. Cl. A61B 5/1459; A61B 5/00; A61B 5/145

(52) U.S. Cl. CPC A61B 5/1459; A61B 5/14514; A61B 5/685

(57) ABSTRACT

A manufacture for assaying a first analyte from interstitial fluid includes hydrogel needles that draw up the interstitial fluid when inserted through a patient's skin, a paper substrate in fluid communication with the needles to receive the interstitial fluid, and a detector. The paper substrate includes a sampling zone that receives the interstitial fluid from the needles. The detector has been functionalized to provide a visual indicator in response to exposure to the first analyte.

### RELATED APPLICATION

This application claims priority from U.S. Patent Application No. 63/773,744, filed Mar. 18, 2025, the entire disclosure of which is incorporated herein by reference.

### GOVERNMENT SUPPORT

This invention was made with government support under W81XWH-21-2-0012 awarded by the United States Army. The government has certain rights in the invention.

### BACKGROUND

Human C-reactive protein (CRP) is a key early clinical indicator of inflammation due to infection and other medical conditions. Additionally, it serves as a main cytokine biomarker for the diagnosis and prognosis of sepsis.

The liver synthesizes C-reactive protein. From there, the bloodstream transports it to the inflamed region. At times the concentration of C-reactive protein in the inflamed region becomes too high. This can cause significant damage.

Another example arises because of chronic stress' tendency to promote abnormal secretion of cortisol and dopamine. This can significantly affect both physical and mental health.

Cortisol, the primary hormone associated with stress, typically exhibits a diurnal rhythm. Its concentration peaks in the morning and decreases throughout the day. However, chronic stress can disrupt this pattern. This can result in persistently elevated or erratic fluctuations in cortisol levels. This, in turn, can contribute to conditions such as hypertension, immune dysfunction, and metabolic disorders. Dopamine is an essential neurotransmitter involved in mood regulation and reward pathways. It too can exhibit dysregulated secretion under chronic stress. This can lead to mood disorders including depression, anxiety, and cognitive impairments.

### SUMMARY

In another aspect, the invention features a manufacture for assaying a first analyte from interstitial fluid. Such a manufacture includes hydrogel needles that draw up the interstitial fluid when inserted through a patient's skin, a paper substrate in fluid communication with the needles to receive the interstitial fluid, and a detector. The paper substrate includes a sampling zone that receives the interstitial fluid from the needles. The detector has been functionalized to provide a visual indicator in response to exposure to the first analyte.

In some embodiments, the manufacture also includes a clamshell housing. This clamshell housing includes a sampling side, from which the needles protrude, a detection side, and a fold line that separates the sampling side from the detection side. The sampling side includes the sampling zone and the detection side includes the detector.

In other embodiments, the detector includes a molecularly imprinted polymer and carbon nanodots. The molecularly imprinted polymer is configured to bind to the first analyte. This binding of the first analyte causes a change in fluorescence of the carbon nanodots. The resulting change in fluorescence is the visual indicator.

Also among the embodiments are those in which the first analyte is selected to be C-reactive protein.

In some embodiments, the detector includes a first detection zone and a second detection zone. The first and second detections zones extends from proximal to distal ends thereof. Both proximal ends are in fluid communication with the sample zone. The first detection zone is functionalized to change color in response to exposure to the first analyte and the second detection zone is functionalized to change color in response to exposure to a second analyte, which differs from the first analyte.

Among the foregoing embodiments are those in which the first analyte is cortisol the second analyte is dopamine. These embodiments also include those in which the first detection zone is functionalized with tetramethylammonium hydroxide and the second detection zone is functionalized with a tetravalent cation of cerium.

In another aspect, the invention features a sampling patch for assaying an analyte from interstitial fluid. The patch includes a clamshell housing and needles. The clamshell housing comprises a sampling side, a detection side, and a fold line that separates the sampling side from the detection side. The needles are hydrogel needles that protrude from the sampling side. The sampling side comprises a paper substrate that is in fluid communication with the hydrogel needles. This promotes transport of the interstitial fluid through the hydrogel needles and towards the paper substrate. The detection side of the housing is configured to provide a visual indicator of a concentration of the analyte.

In some embodiments, the detection side comprises a molecularly imprinted polymer and carbon nanodots, wherein the molecularly imprinted polymer is configured to bind the analyte and wherein binding of the analyte causes a change in fluorescence of the carbon nanodots, wherein the visual indicator is the change in fluorescence.

In other embodiments, the analyte is selected to be C-reactive protein.

In another aspect, the invention features a manufacture for quantifying first and second analytes in interstitial fluid. The manufacture includes needles, specifically hydrogel microneedles, and a paper substrate. The paper substrate comprises a sample zone, a first detection zone, and a second detection zone. The first detection zone extends from a proximal end to a distal end thereof. Similarly, the second detection zone extends from a proximal end to a distal end thereof. Both proximal ends are in fluid communication with the sample zone, as are the needles. The first detection zone is functionalized to change color in response to exposure to the first analyte. Similarly, the second detection zone is functionalized to change color in response to exposure to the second analyte.

Embodiments include those in which the two analytes are cortisol and dopamine.

Other embodiments include those in which the first detection zone is functionalized with tetramethylammonium hydroxide and the second detection zone is functionalized with a tetravalent cation of cerium.

### BRIEF DESCRIPTION OF THE DRAWINGS

FIG. 1 shows a sampling patch for assaying C-reactive protein in interstitial fluid and

FIG. 2 shows a sampling patch for monitoring two analytes obtained from interstitial fluid.

### DETAILED DESCRIPTION

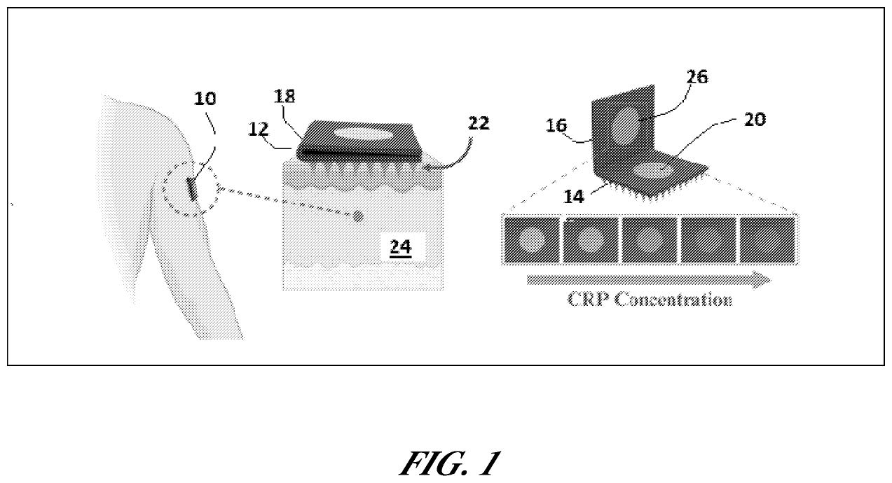

FIG. 1 shows a sampling patch 10 for assaying C-reactive protein comprises a bivalve clamshell housing 12 having a sampling side 14 and a detection side 16. A fold line 18 separates the sampling side 14 and the detection side 16.

The sampling side 14 includes a sampling zone 20 made of a paper substrate. This sampling zone 20 contacts proximal ends of plural hydrogel needles 22. Each needle 22 extends away from the sampling side 14 and terminates at a distal end thereof. The distal ends of the needles 22 penetrate a patient's skin 22 and thus draw interstitial fluid therefrom. The proximal ends of the needles 22 are in fluid communication with the sampling zone 20. As a result, interstitial fluid drawn by the needles 22 reaches the sampling zone 20.

The detection side 16 features a paper substrate on which is formed a detection zone 28 that comprises molecularly imprinted polymers and carbon nanodots integrated therein. These are selected such that binding of the C-reactive protein to the polymer tends to quench fluorescence of the carbon nanodots to an extent that depends on the concentration of the analyte, an example of which is C-reactive protein.

An assay for the analyte begins with folding the housing 12 along its fold line 18 so that the sample zone 20 and detection zone 28 come into contact. The housing 12 is then pressed onto the skin 22 to allow the hydrogel needles 22 to access the interstitial fluid. After a brief sampling interval to allow interstitial fluid to flow from the distal ends of the needles 22 to the sampling zone 20 and onward to the detection zone 26, the process continues with removing the patch 10 and unfolding the housing 12 thereby detaching the sample zone 20 and the detection zone 26 from each other and permitting visual inspection of the detection zone 26.

Binding of the C-reactive protein to the molecularly imprinted polymer quenches the fluorescence of the carbon nanodots as a result of surface adsorption and phot-induced electron transfer. The extent to which such quenching occurs depends on the concentration of C-reactive protein. As a result, by observing the fluorescence, it is possible to infer the concentration of C-reactive protein by observing the extent of fluorescence in the sample zone 20.

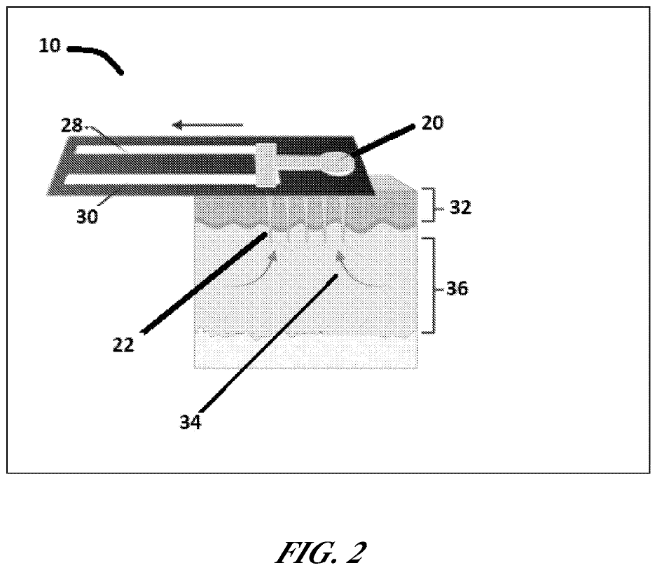

FIG. 2 shows an alternative sampling patch 10 having a sample zone 20, a first detection zone 28, and a second detection zone 30, both of which are in fluid communication with the sample zone 12. Each detection zone 28, 30 has a proximal end that begins at the sample zone 12. Each detection zone 28, 30 extends distally away from this proximal end.

The first and second detection zones 28, 30 are functionalized with first and second reagents to detect first and second analytes. The sample zone 20 and the first and second detection zones 28, 30 are formed on a paper substrate. As a result, interstitial fluid in the sample zone 20 wicks its way into the detection zones 28, 30.

The sampling patch 10 also includes hydrogel needles 22, each of which has a proximal end in fluid communication with the sample zone 20 and a distal end that penetrates the epidermis 32 so as to be placed in contact with interstitial fluid 34 in the dermis 36.

In operation, the needles 22 penetrate the skin. This causes transport of interstitial fluid 34 from beneath the skin towards the sample zone 20. This transport also brings first and second target analytes with it.

From the sample zone 20, the interstitial fluid proceeds to the proximal ends of the first and second detection zones 28, 30 and travels distally along the first and second detection zones 28, 30.

As interstitial fluid traverses the first detection zone 28, the first analyte reacts with the first reagent in the first detection zone 28. This causes a segment of the first detection zone 28 to change color. This segment begins at the proximal end and extends distally along the first detection zone 28 by some length that can be measured. The length of this colored segment provides a basis for inferring the concentration of the first analyte.

Similarly, as interstitial fluid 34 traverses the second detection zone 30, the second analyte reacts with the second reagent in the second detection zone 30. This causes a segment of the second detection zone 30 to change color. This segment begins at the proximal end and extends distally along the second detection zone 30 by some length that can be measured. The length of this colored segment provides a basis for inferring the concentration of the second analyte.

In some embodiments, the first and second analytes are cortisol and dopamine, respectively. Among these embodiments are those in which the first and second reagents are tetramethylammonium hydroxide (TMAOH)/blue tetrazolium and tetravalent cerium cation (Ce⁴+) solutions to cause a colorimetric response to the presence of cortisol and dopamine, respectively.

Further details concerning the patch 10 and its manufacture are in the attached appendices, the contents of which are incorporated herein by reference.

What is claimed is:

1. A manufacture for assaying a first analyte from interstitial fluid, said manufacture comprising needles that draw up said interstitial fluid when inserted through a patient's skin, a paper substrate in fluid communication with said needles to receive said interstitial fluid, said paper substrate comprising a sampling zone that receives said interstitial fluid from said needles, and a detector, wherein said detector has been functionalized to provide a visual indicator in response to exposure to said first analyte and wherein said needles are hydrogel needles.

2. The manufacture of claim 1, further comprising a clamshell housing, wherein said clamshell housing comprises a sampling side, a detection side, and a fold line that separates said sampling side from said detection side, wherein said needles protrude from said sampling side, wherein said sampling side comprises said sampling zone, and wherein said detection side of said housing comprises said detector.

3. The manufacture of claim 1, wherein said detector comprises a molecularly imprinted polymer and carbon nanodots, wherein said molecularly imprinted polymer is configured to bind to said first analyte, wherein binding of said first analyte causes a change in fluorescence of said carbon nanodots, wherein said visual indicator is said change in fluorescence.

4. The manufacture of claim 1, wherein said first analyte is selected to be C-reactive protein.

5. The manufacture of claim 1, wherein said detector comprises a first detection zone and a second detection zone, wherein said first detection zone extends from a proximal end to a distal end thereof, wherein said second detection zone extends from a proximal end to a distal end thereof, wherein said proximal ends of said first and second detection zones are in fluid communication with said sample zone, wherein said first detection zone is functionalized to change color in response to exposure to said first analyte, and wherein said second detection zone is functionalized to change color in response to exposure to a second analyte.

6. The manufacture of claim 4, wherein said first analyte is cortisol and wherein said second analyte is dopamine.

7. The manufacture of claim 4, wherein said first detection zone is functionalized with tetramethylammonium hydroxide and said second detection zone is functionalized with a tetravalent cation of cerium.
